(19) United States
(12) Patent Application Publication
(10) Pub. No.: US 2020/0139009 A1
(43) Pub. Date: May 7, 2020

(54) SELF-ASSEMBLY DERIVED CO-CONTINUOUS MATERIALS FOR BIOMEDICAL DEVICES

(71) Applicant: THE REGENTS OF THE UNIVERSITY OF CALIFORNIA, Oakland, CA (US)

(72) Inventors: Ali Mohraz, Irvine, CA (US); Todd J. Thorson, Irvine, CA (US); Elliot L. Botvinick, Irvine, CA (US)

(21) Appl. No.: 16/625,213

(22) PCT Filed: Jun. 8, 2018
(86) PCT No.: PCT/US2018/036787

§ 371 Date: Dec. 20, 2019

Related U.S. Application Data
(60) Provisional application No. 62/522,590, filed on Jun. 20, 2017.

(51) Int. Cl. A61L 27/34; A61L 27/52

(52) U.S. Cl. CPC A61L 27/34; A61L 27/52; A61L 2420/06; A61L 2420/04; A61L 2400/12

(57) ABSTRACT

A method and material composition of a highly porous material that is applied to an object, such as a biomaterial implant and biomedical device, is described. The method involves forming a bijel mixture that is exposed to at least an outer surface of an object. Thereafter, a precursor is added to the bijel mixture to allow the precursor to transport into a particular liquid phase of the bijel mixture. After at least partial transport, the precursor-containing liquid phase of the bijel mixture is solidified to form a bijel-templated material (BTM) that is bonded to a surface of the object.

### CROSS-REFERENCE TO RELATED APPLICATIONS

This application claims the benefit of priority on U.S. Provisional Application No. 62/522,590 filed Jun. 20, 2017, the entire contents of which are incorporated by reference.

### NOTICE OF GOVERNMENT-SPONSORED RESEARCH

This invention was made with Government support under Grant No. NNX13AQ69G, awarded by NASA Research Opportunities in Complex Fluids and Macromolecular Biophysics. The Government has certain rights in the invention.

### FIELD

One embodiment of the disclosure relates to a material for improving the biocompatibility of devices. More specifically, an embodiment of the disclosure relates to a polymer, such as a hydrogel for example, for coating medical implants or at least certain portions of medical devices inserted into tissue.

### BACKGROUND

The body's reaction to foreign materials is a significant factor in the development of successful implantable biomaterials and biomedical devices including continuous glucose sensors, prosthetics, catheters, pace maker leads, and tissue regeneration materials. Upon implantation, the normal wound healing process is initiated due to local tissue damage, and the fate of the implant is contingent on the host-material interaction or foreign body response (FBR). In the worst-case scenario, proteins quickly adsorb on the implant material surface and blood platelets develop a clot at the interface. From here, early immune cells (neutrophils) assess the inflammatory response and recruit macrophage reinforcements through the release of chemical signals. Eventually, fibroblasts spread and deposit collagen at the host-material interface. The resulting fibrosis will encapsulate the implant, thus destroying the intended functionality of the biomaterial or biomedical device.

The down-regulation of inflammatory signals and mitigation of the FBR in biomaterials has been a subject of study for decades. Many implants developed from naturally derived and synthetic polymers (e.g. chitosan and polyethylene glycol “PEG”, respectively) are considered safe for implantation due their lack of toxic degradation products and their chemically inert surfaces.

It has been shown that controlling the host-material interface requires distinct architectural cues, not just chemical compatibility. Materials with physical features on the order of cellular dimensions, such as 10-30 microns (μm) for example, display a higher degree of acceptance by the host (physiological) tissue. Physiologically, researchers have investigated the FBR-related tissue response relative to implant morphology and determined (1) curved pore features with cellular-equivalent dimensions mitigate the ability of early immune cell spreading associated with heightened inflammatory signaling and (2) host cells interacting with curved material micro-features are inhibited from advanced collagen remodeling and formation of a dense fibrotic capsule at the host-material interface.

One type of conventional FBR dampening material has been created using precise, sphere-templating methods in which a continuous polymer is formed around densely packed particles (D-50 μm), and the particles are subsequently removed to create the porous material. Such materials feature spherical void pockets with adjustable sizes (e.g., 20-90 μm) with narrow connections between adjacent pockets. The FBR mitigating capability of this material exhibited disadvantages contrary to the goals of any tissue intermediary material.

Specifically, a first disadvantage is that the uniform spherical pore architecture is not an optimal solution in dampening the FBR. In fact, fibrotic FBR may be observed in many cases after the implant has dwelled in host tissue for at least two weeks. While the surface of the material is curved inside spherical pore domains, these pore domains are connected via tight pore windows. Furthermore, the material edges are sharp due to the particle templating process used. These morphological features may contribute to a heightened inflammatory response by the host tissue after implantation thereby limiting the efficacy for mitigating the FBR and thus the dwelling lifetime of the implant.

A second disadvantage associated with conventional materials having spherical pores is the tendency of the host tissue to penetrate the narrow windows between spherical pores. Hence, vascularization of the implant is limited by the narrow pore windows.

A third disadvantage associated with the conventional materials having spherical pores is scalability in the fabrication of products implementing such materials. The conventional particle templating process must employ sonication to carefully arrange the particles into close-packed layers, and then sintering spheres in these layers to partially fuse to ensure pore interconnectivity. A volume between the spheres is then filled with a precursor followed a polymerization process to convert the precursor into a polymer after which the particles must then be removed. This whole process is time-consuming, costly, and limits the scalability of the product.

### BRIEF DESCRIPTION OF THE DRAWINGS

Embodiments of the disclosure are illustrated by way of example and not by way of limitation in the figures of the accompanying drawings, in which like references indicate similar elements and in which:

FIGS. 1A-1B are exemplary representative diagrams of forming a bicontinuous interfacially jammed emulsion gel (bijel).

FIG. 2 is an exemplary diagram of the synthesis procedure for creating bijel-templated material (BTM).

FIGS. 3A-3B are exemplary renderings of a computed tomography (CT) scan of one embodiment of a bijel-templated material formed illustrating continuous paths within the void domain.

FIG. 4A is a first exemplary illustration of the bijel-templated material coating process.

FIG. 4B is a second exemplary illustration of the bijel-templated material coating process.

FIG. 4C is a third exemplary illustration of the bijel-templated material coating process.

FIGS. 5A-5I are illustrations of in vivo histological results following implantation of bijel-templated materials

FIG. 6A is an illustration of etched polytetrafluoroethylene (PTFE) bonded to non-bijel-templated polyethylene glycol (PEG).

FIGS. 6B-6C are illustrations of etched polytetrafluoroethylene (PTFE) bonded to bijel-templated polyethylene glycol (PEG).

### DETAILED DESCRIPTION

In general, embodiments of the disclosure describe a technique to create highly porous materials for biomaterial implants and biomedical devices. The architecture of this material features fully penetrating, non-constricting, curved channels with predominantly negative Gaussian curvature on channel walls throughout the material.

Herein, the terms “or” and “and/or” as used should be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive

### I. General Overview

According to one embodiment of the disclosure, this technique is adapted to generate a unique morphology within materials used in implantable devices and cell therapy procedures, where the unique morphology provides a unique class of implantable materials derived from a self-assembly process. A mixture is first formed with at least two liquids that feature a miscibility gap, and colloidal particles (e.g., nanoparticles). In one example, the two liquids may include water and 2,6-lutidine, and the colloidal particles may include silica particles. The silica particles may be doped with fluorescent molecules such as Rhodamine or Fluorescein. Next, for this embodiment, the mixture is heated and self-assembles into a bijel. A material precursor of choice is then introduced into one of the liquid phases of the bijel, such as oil or water. The precursor-containing liquid phase is solidified to preserve the architecture of the bijel forming what is termed a “bijel-templated material” (hereafter, “BTM”). According to one embodiment, ultraviolet light polymerizes the precursor-containing liquid phase to form the BTM. The remaining liquid phase remains or may be drained.

The BTM features a unique, channel structure suitable for implantation. The channel structure comprises a penetrating network of curved channels that may resemble a labyrinth-like network of connected paths. The consistent curvature over the entirety of these channels, including where the channels terminate at the surface of the material, may promote dampened host cell signaling thereby potentially reducing the formation of a dense avascular tissue layer at the host-material interface. Additionally, or the alternative, the BTM may potentially induce the formation of new vessels at the surface of and within the volume of the material. Furthermore, the interconnecting channel network provides non-constricting paths for newly formed blood vessels to form throughout the volume of the material, as well as optimal transport properties for the exchange of nutrients and waste products between the vasculature and/or tissue and the material.

The BTM formation process, which includes the bijel self-assembly process described herein, can be used to make a variety of materials with varying properties and surface chemistries. Stated differently, this process provides a platform for generating BTMs comprised of different materials for tailoring properties of a selected BTM to a specific biomedical application. Hence, the BTM formation process is not limited to one biomedical application, but rather is flexible and can be tailored to a variety of biomedical applications.

Lastly, the unique microstructure of a BTM is formulated by an accelerated process with a lesser number of operations as compared the formation of inferior conventional FBR-mitigating materials. This accelerated process enables large scale manufacturing of BTM-based products.

### II General BTM Composition

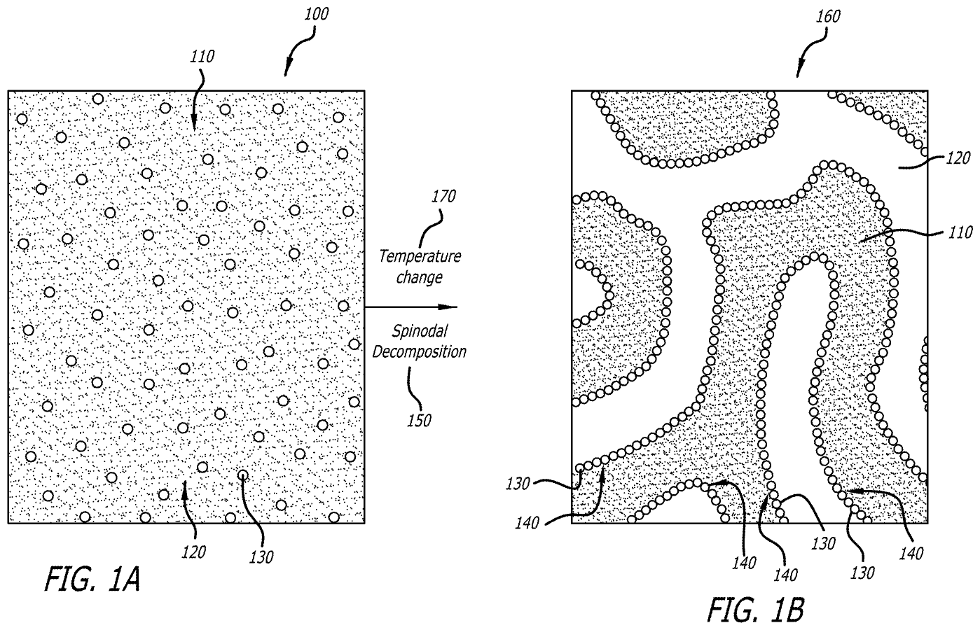

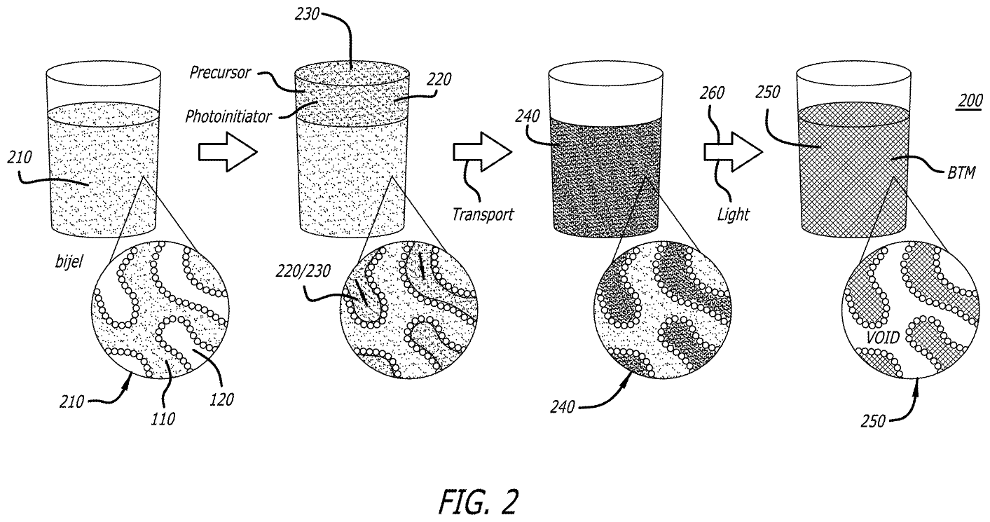

Referring now to FIGS. 1A-1B and FIG. 2, illustrative embodiments of the creation of bijels and BTMs are shown. The bijels and BTMs are based on a plurality of components. In general, bijels are a class of soft materials comprised of uniquely assembled interpenetrating liquid phases. BTMs may comprise a polymer domain (FIG. 2), where the polymer domain is templated by a bijel resulting in a BTM.

In particular, as shown in FIGS. 1A-1B, an exemplary embodiment of the unique morphology of bijels is shown. The formation of a bijel 160 begins with a mixture 100 that includes a first liquid (e.g., hydrophilic liquid) 110, a second liquid 120 (e.g., liquid partially miscible with the first liquid) different than the first liquid 110, and neutrally wetting colloidal particles 130 (FIG. 1A). Formation of a bijel 160 occurs through arrested phase separation of the mixture 100 undergoing spinodal decomposition 150 in the presence of neutrally wetting colloidal particles 130. During phase separation, the particles 130 adsorb to the liquid-liquid interface 140, and the system becomes jammed as the interfacial area is sufficiently reduced to just accommodate particles 130, as shown in FIG. 1B. The resulting soft material is comprised of two bi-continuous, fully interpenetrating liquid domains. The internal local curvature of both resulting liquid domains can be tuned over the range of 5 m to 850 m solely through the volume fraction of particles in the system. The upper limit is governed by density differences between the two liquids 110 and 120 that tend to macroscopically separate the first liquid 110 from the second liquid 120.

In bijel formation, as shown in FIG. 1A, the first liquid 110 (e.g., purified water) is added to a predetermined amount of colloidal particles 130, where the particles are dispersed within the first liquid 110. Next, a bijel mixture is formed by adding the second liquid 120 (e.g., lutidine such as 2,6-lutidine) to the first liquid 110 including the colloidal particles 130. The second liquid 120 is different from and is partially miscible with the first liquid 110. According to one embodiment the disclosure, the bijel mixture undergoes a change in temperature (e.g., increased application of heat for a selected period of time). The temperature change 170 brings the bijel mixture to or past a critical temperature and induces spinodal decomposition phase separation 150 between the first liquid 110 and the second liquid 120 such that the colloidal particles 130 operate as the liquid-liquid interface 140 illustrated in FIG. 1B. This stabilizes the resulting bijel 160. The bijel 160 may be further stabilized through additional heat transfer.

More specifically, according to specific embodiment of bijel formation, fluorescently labeled (Rhodamine B) silica particles may be carefully dried in a vacuum oven to achieve neutral degree of wetting with both liquids (e.g., water and 2,6-lutidine). Next, a predetermined mass of nanoparticles is measured and deposited into a glass scintillation vial. Purified (Millipore) water is added to the vial and particles are dispersed. One dispersion technique involves an use of an ultrasonic horn and bath (2 minutes each, 2 cycles). Next, a mixture is formed by adding 2,6-lutidine (6.4 mol % 2,6-lutidine) to the dispersion in a glass tube, vortex mixed for 10 seconds, and pipetted into a second glass tube. This tube containing the mixture is immediately placed into a heating device for a selected period of time (e.g., in microwave for approximately 30 seconds at a low power setting). A predetermined amount of supplied heat brings the mixture to a critical temperature and induces spinodal decomposition phase separation between the water and the 2,6-lutidine such that the nanoparticles adsorb to the liquid-liquid interface as described above with an exemplary formation shown in FIG. 1B, thus kinetically stabilizing the resulting bijel. The characteristic morphological features of the bijel 160, which include co-continuous, fully penetrating liquid domains separated by a particle monolayer exhibiting continuous negative and zero mean curvature, arise from the minimal surface process of spinodal decomposition. The bijel is then stabilized by additional heating within an oven with temperature maintained at approximately 70° C.

Referring now to FIG. 2, an exemplary diagram of the synthesis procedure 200 for creating bijel-templated material (BTM) 250 is shown. In the formation of the BTM 250, the kinetically stable bijel 210 (previously illustrated as bijel 160 including the first liquid 110 and the second liquid 120 of FIG. 1B) can be used to template a polymer/void construct by exploiting the incompatible chemistries of the two liquid domains and selectively polymerizing one liquid phase. Briefly, a precursor 220 (e.g., monomer or material precursor) which may also be mixed with a substance 230 (e.g., photoinitiator that creates a reactive liquid phase) is placed on top of the bijel 210 and allowed to transport (e.g., diffuse) preferentially into one of the liquid domains, as dictated by the precursor solubility within each phase.

As further shown, according to this embodiment of the disclosure, the BTM 250 may be formed by photopolymerization of the precursor-containing liquid phase, in response to exposure of the photoinitiator 230 within the bijel 240 to the appropriate wavelength and dosage of light 260. After photopolymerization, if necessary, any excess polymer not exhibiting the bijel-templated morphology may be removed and unreacted materials may be removed through washing with isopropyl alcohol or other suitable solutions. Alternatively, although not shown in detail, the BTM 250 may be formed by another type of polymerization in lieu of the application of light 260 (e.g., thermally activated, chemically activated, time-based activation, or irradiation) based on the type of precursor 220 added to the bijel 240 and/or type of substance 230 added.

In one embodiment of the disclosure, the bijel 210 is formed from a solution of water and 2,6-lutidine at the critical composition (6.4 mol % 2,6-lutidine) and Rhodamine B-labeled silica particles (D-500 nm). A BTM is formed from the bijel by introducing a hydrophobic monomer 220 (e.g., polyethylene glycol diacrylate (PEGDA Mₙ:258)) mixed with an oil soluble photoinitiator 230 (e.g., Darocur® 1173). The hydrophobic monomer-containing liquid phase is then polymerized by exposure to light 260 as described above, and if required, the remaining liquid phase is drained. Silica particles may subsequently be removed through an acid or base etch leaving only the cross-linked polymer. Rhodamine B may also adsorb to the polymer as molecules become liberated during the etching process. Any remaining Rhodamine B can be degraded by applying a potassium persulfate solution and subsequent exposure to ultraviolet radiation.

In accordance with another embodiment, a BTM can be formed from the kinetically stable bijel by exploiting the incompatible chemistries of the two liquid domains and selectively replacing (partially or entirely) at least one of the liquid domains with an alternative material. For example, a liquid not having optimal characteristics for the formation of a bijel, may be integrated into the bijel following particle jamming and stabilization. In another embodiment, a monomer or material precursor mixed with a photoinitiator may be placed on top of the bijel and allowed to transport preferentially into one of the liquid domains, as dictated by the precursor solubility within each phase.

A wide variety of precursors may be used to create bijel-templated materials as long as the precursor is solely solubilized by one of the liquids of the bijel, each liquid of the bijel can either be one of the liquids used to form the bijel, or a liquid subsequently replacing (either partially or in part) one of the liquids used to form the bijel. The precursors may contain a polymerizable component. BTMs may comprise biocompatible materials including, but are not limited or restricted to, polyethylene glycol (PEG), poly(hydroxyethylmethacrylate) (PHEMA), polycaprolactone (PCL), and polylactide (PLA). Furthermore, a BTM (e.g. one made of PEG) may be used as a skeletal structure available for the casting of additional materials. These materials may include, but are not limited to, zwitterionic hydrogels comprised of poly(carboxybetaine methacrylate (PCBMA), PDMS, poly(N-vinylpyrrolidone) (PVPON), poly(N-isopropylacrylamide) (PNIPAM), polytetrafluoroethylene (PTFE), or copolymers containing biodegradable or photodegradable blocks.

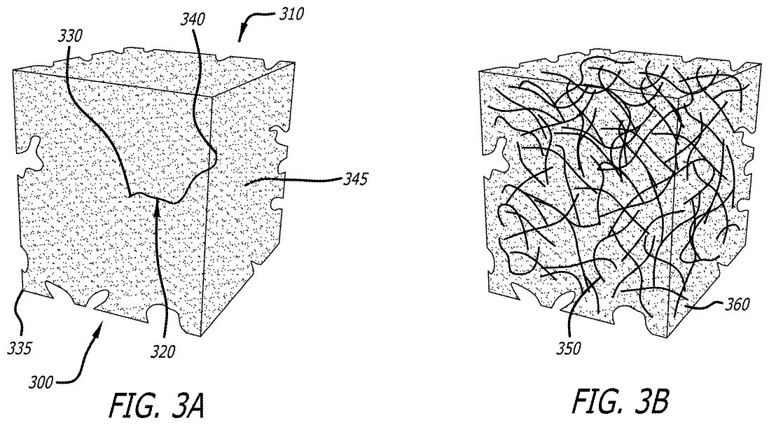

Referring to FIGS. 3A-3B, the resultant polymer morphology can be imaged using various imaging modalities such as digital microscopy, scanning electron microscopy (SEM), and computed tomography (CT). High resolution three-dimensional renderings obtained via CT permit detailed analysis of the unique morphology imparted onto a BTM 310, including fully penetrating, non-constricting, curved channels in the void domain.

Herein, the three dimensional structure 300 of the BTM 310 (in this case made from PEG precursor and formed into a cubic structure from which the water phase has been drained) has been imaged by CT and rendered. The shortest continuous path 320 between a first opening 330 of the void domain at one surface 335 of the BTM 310 and a second opening 340 of the void domain located at another (e.g. opposite) surface 345 of the BTM 310 can be computed from CT scans as shown in FIG. 3A. Further, as illustrated in FIG. 3B, the CT scan can be used to calculate all possible continuous paths 350 throughout the entire polymer volume 360. These results exhibit the inherent and unique connectivity of the void domains, and similar results are found when computing shortest continuous paths within the polymer domain.

### III Detailed Description of BTM Coating Process

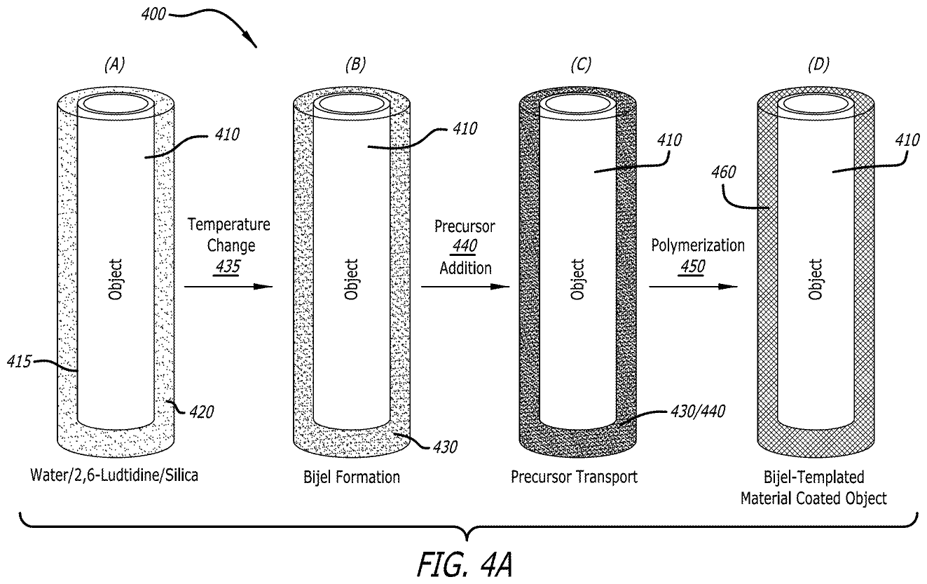

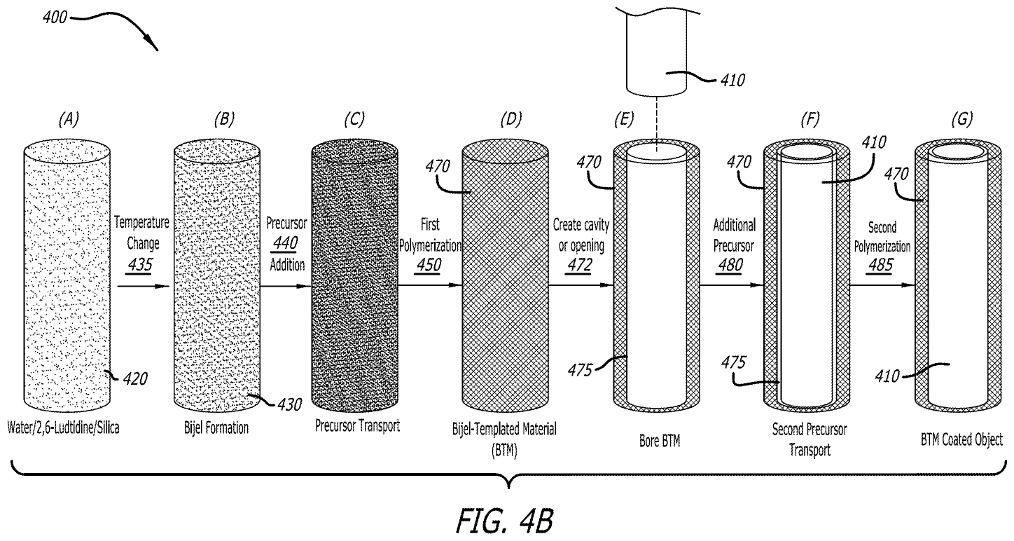

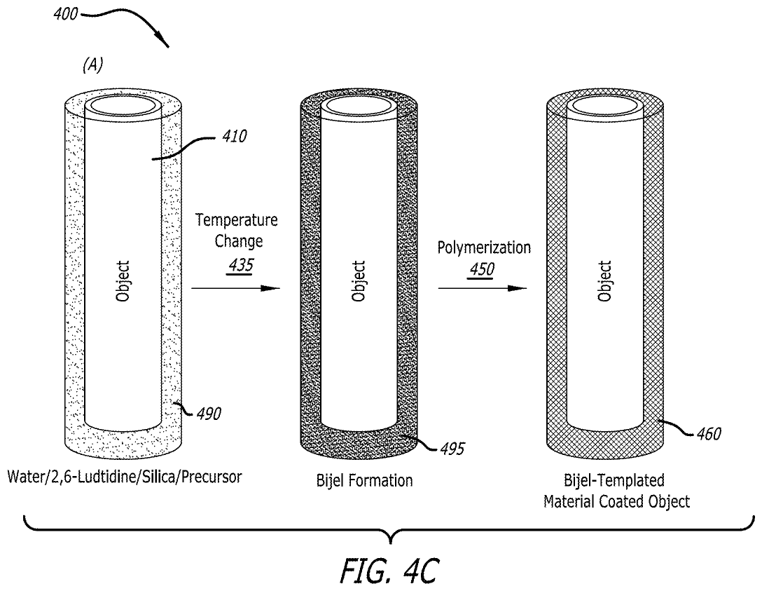

Referring now to FIG. 4A, a first exemplary illustration of the bijel-templated material coating process is shown. Herein, a portion or entirety of a biomedical implant or other device (hereinafter, “object” 410) designed to be inserted or implanted into the body may be coated with BTM 460 according to (but not limited or restricted to) the below-described processes. The BTM coating 460 may be bonded to a surface of the object 410, providing that the surface receiving the BTM coating 460 facilitates bonding, either naturally or via functionalization. An example of the above-identified object 410 may include, but is not limited or restricted to, transcutaneous cannula and drug infusion port, intravenous catheter and access port, artificial organ device (e.g. artificial pancreas), one or more analyte sensors, a prosthetic, and/or cell therapy/drug delivery implant. Materials that may be coated with BTMs include, but are not limited to, PTFE (after removing a fraction of fluorine through common etching techniques) and surface activated variants of polyvinyl chloride (PVC), polyurethane (PU), PDMS, polyether ether ketone (PEEK), or polyethylene.

Referring still to FIG. 4A, an exemplary illustration of a first embodiment of the BTM coating process 400 is shown. In this embodiment, in process step (A), the object 410 is a PTFE tube, which has been treated with sodium naphthalene on its outer surface 415 to allow for covalent bonding to the BTM. The sodium naphthalene treatment is commonly employed industrially to etch PTFE tubing surfaces. Fluorine atoms are removed during the etching process leaving chemical groups available for bonding through radical polymerization of acrylate-containing monomers or material precursors. The object 410 (e.g., an etched PTFE tube) is placed in the first liquid/second liquid/colloidal (e.g., water/2,6-lutidine/silica) mixture 420. As shown in process step (B), the bijel 430 is next formed around the object 410 by a change in temperature 435 of the system (e.g., a raise of temperature of the mixture 420 above the critical temperature of approximately 34.1° C.).

Next, in process step (C), a desired precursor 440 (e.g., a monomer or material precursor), is added to the system and sufficient time is allotted (e.g., 2-4 hours) to allow transport of the precursor 440 into one of the phases (e.g., 2,6-lutidine rich phase) of the bijel 430. In one embodiment, the precursor 440 is composed of PEGDA and a photoinitator. For this embodiment, as shown in process (D), photopolymerization 450 forms the BTM 460 and bonds the PEG phase of the BTM 460 to the object 410 (e.g., PTFE tube). In lieu of conducting photopolymerization to form the BTM 460, another type of polymerization (e.g., thermal, chemical, time-based, or another type of irradiation) may be used with a suitable precursor 440 being added to the system.

Although not shown, after photopolymerization, if desired, any excess polymer not exhibiting the bijel-templated morphology may be removed and unreacted materials may be removed through washing with suitable solvents. In one embodiment, the bijel is formed from a solution of water and 2,6-lutidine at the critical composition (6.4 mol % 2,6-lutidine) and Rhodamine B-labeled silica particles (D-500 nm). The BTM 460 is formed from the bijel 430 by introducing a hydrophobic monomer (polyethylene glycol diacrylate (PEGDA Mₙ:258)) mixed with an oil soluble photoinitiator (Darocur® 1173). The hydrophobic monomer-containing liquid phase is then polymerized by exposure to light as described above, and if required, the remaining liquid phase is drained. Silica particles may subsequently be removed through an acid or base etch leaving only the cross-linked polymer. Rhodamine B may also adsorb to the polymer as molecules become liberated during the etching process. Any remaining Rhodamine B can be degraded by applying a potassium persulfate solution and subsequent exposure to ultraviolet radiation.

Referring now to FIG. 4B, in a second embodiment of the BTM coating process, a BTM block 470 is created in accordance with a similar process as set forth in FIG. 4A. Herein, the bijel 430 is formed from the first liquid/second liquid/colloidal particles (e.g., water/2,6-lutidine/silica) mixture 420 by applying a change in temperature 435 to the system (e.g., a raise of temperature of the mixture 420) as set forth in process steps (A-B). However, no object is placed within the mixture 420. As previously described, and is illustrated in process step (C), the desired precursor 440 is added to the system and sufficient time is allotted (e.g., 2-4 hours) to allow transport of the precursor 440 into the second liquid phase (e.g., 2,6-lutidine rich phase) of the bijel 430 where a first polymerization 450 is utilized to form a BTM block 470 as shown in process steps (C-D). Hence, this process may be used to create BTM blocks without the presence of an object.

As an optional sub-process, as shown in process step (E), portion of the BTM block 470 is removed 472 from the BTM block 470 to create a cavity or an opening 475 propagating through the BTM block. The object 410 may be optionally placed into the cavity or opening 475. For the purpose of binding the object 410 to the BTM block 470, as shown in process step (F), an additional precursor/photoinitiator solution 480 may be added to the polymer phase of the BTM block 470 at a volume sufficient to wet the polymer phase of the BTM block 470. Thereafter, as shown in process step (G), a second polymerization operation 485 is conducted resulting in two effects. First, the second precursor 480 is bonding to the existing polymer phase of the BTM block 470, and second, the BTM bonds directly to the surface of the object 410.

Referring to now FIG. 4C, in a third embodiment of the BTM coating process, the precursor 440 (e.g., monomer or material precursor) is added directly to the water/2,6-lutidine/colloidal particle mixture 490 before forming the bijel, as shown in process step (A). Next, the mixture is applied to the object 410, by either insertion of the object 410 into the mixture 490 (as shown), or application of the mixture 490 onto the object 410 (not shown). Thereafter, bijel formation is carried out as described above. The precursor-containing liquid phase is polymerized and bonded to the object 410 by polymerization (e.g., photopolymerization) to form BTM 460. Removal of excess materials, including nanoparticles, may be performed as described above.

Although not shown, in a forth embodiment of the BTM coating process, the BTM (block) can first be formed as illustrated in FIG. 4C. Next, a portion of the BTM is removed to create an opening propagating through the block or a cavity, and the object is placed into the opening or cavity as described in the second embodiment of the BTM coating process. As previously described above, for the purpose of binding the object to the block, an additional precursor/photoinitiator solution may be added to the polymer phase of the BTM at a volume sufficient to wet the polymer phase of the BTM. A second photopolymerization operation is conducted resulting in two effects. First, the second precursor is bonding to the existing polymer phase, and second, the BTM bonds directly to the surface of the object. Removal of excess materials, including nanoparticles, may be performed as described above.

### IV. Summary of Certain Unique Aspects of the Invention

Self-Assembly Based Formation of BTM.

The process described by this disclosure to form a bijel can be completed rapidly (in seconds) with a minimal number of steps and the bijel then can be used to template a BTM for use in coating an implant or medical device.

Non-Constricting, Fully Penetrating Curved Channel Architecture.

A feature of this invention is the fabrication of BTMs having uniform micro-channel geometry that also results in a network of fully penetrating, non-constricting voids that resemble an extensive labyrinth within the volume of the BTM. The consistent curvature over these channels, including where the channels terminate at the surface of the material, may promote pro-healing host cell signaling thereby potentially reducing the formation of a dense avascular tissue layer at the host-material interface.

Versatility in Material Selection and Post-Processing.

The range of monomer or material precursors that may be used to create BTMs is diverse. This property permits screening of various material surface chemistries and properties in tailoring optimal coatings for many biomedical applications. If precursor solubility in a bijel-forming liquid is an issue for a desired material, a skeleton structure can be created from a biologically inert material (e.g. polyethylene glycol) and the desired product can be cast throughout this skeleton structure.

Scalability and Low Cost.

In comparison to the formation of conventional materials with patterned pores, this invention for fabricating and applying BTMs can be performed by less complex processes to reduce expenditure of energy and time, as well as achieving cost savings.

### V. General Description of Some System Embodiments

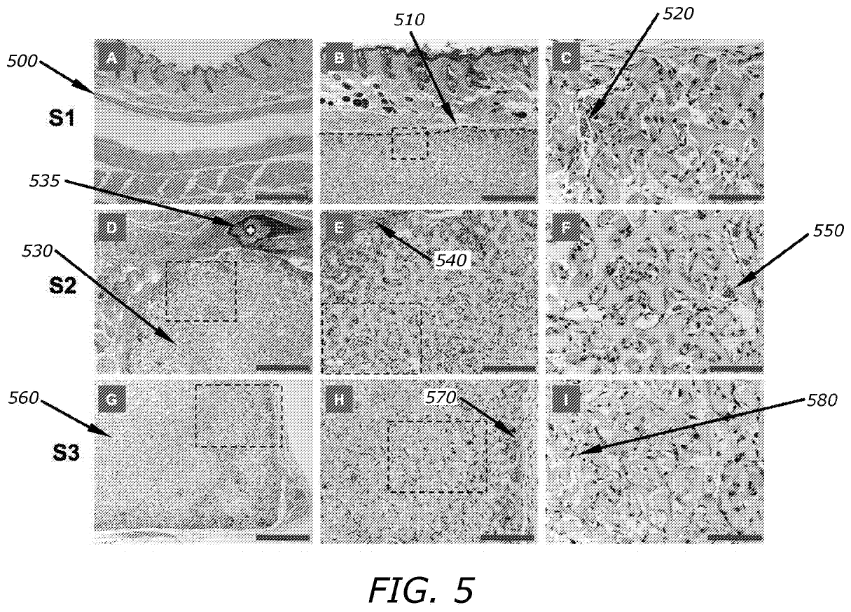

Referring to FIGS. 5A-5I, the immune mitigating ability of bijel-templated morphology is demonstrated for cylindrical PEG-based BTM samples with channel diameters of 21 μm or 30 μm. The samples are implanted in the subcutaneous space in nude (athymic) mice for 28 days. In particular, FIGS. 5A-5I show hematoxylin and eosin (H&E) staining of explanted tissue/implant samples and demonstrates the host tissue invasion and immune response dynamics to a BTM. Magnified regions of interest in micrographs are denoted by the dashed boxes; one sample at increasing magnification is shown per row. Control skin 500 (FIG. 5A) was similar to that surrounding BTM implants (FIG. 5B). A dense fibrotic capsule is not detectable at the tissue-BTM interface (depicted by horizontal dashed line 510 in FIG. 5B). A higher magnification image shows that the BTM is well vascularized 520 near its interface with the skin and as deep as 300 μm from the interface (FIG. 5C). Histology for a second implant 530 (FIG. 5D) was again void of a dense fibrotic capsule (for comparison, note the response to a suture 535), with loosely organized collagen 540 at the interface (FIG. 5E) and perfused blood vessels within the micro-channels 550 as deep as 600 m from the interface (FIG. 5F). Lastly, histology for a third implant 560 shows the BTMs are cleanly separated from the implantation site without adhesions to the tissue, indicating a very weak fibrotic response (FIG. 5G). Of note, only loosely organized collagen 570 was present at the interface (FIG. 5H) and dense vasculature 580 may be observed as deep as 800 m from the tissue interface (FIG. 5H-5I). The results shown here represent successful dampening of the foreign body response with perfused blood vessels deep within the material micro-channels after four weeks.

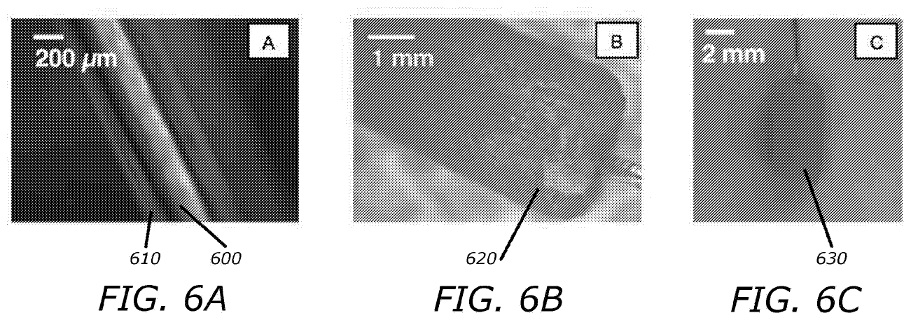

Referring now to FIG. 6A, a wire (e.g. object) 600 coated with etched PTFE 610 was inserted into a precursor solution composed of polyethylene glycol diacrylate (PEGDA Mₙ:258) with 1% v/v Darocur® 1173 photoinitiator. Etched PTFE was supplied by a vendor. Etching of PTFE is accomplished using sodium naphthalene to remove fluorine atoms from the surface of the PTFE coating. Once these fluorine atoms have been removed, the carbon in the PTFE backbone becomes available for bonding of various adhesives. Acrylates will bond to these available carbons during radical polymerization. In the current embodiment, polyethylene glycol diacrylate (PEGDA Mₙ:258) undergoing photo-initiated radical polymerization then bonds to the etched tubing surface.

The PTFE coating successfully bonded to the PEGDA following treatment with ultraviolet light. As now shown in FIGS. 6B-6C, PEGDA-based BTMs 620 are bonded to the etched PTFE coated wire according to processes described above in the second embodiment of the BTM coating process. The PTFE coated wire was successfully bonded to the BTM 630.

1. A method comprising:
forming a bijel mixture that is exposed to at least an outer surface of an object;
adding a precursor to the bijel mixture to allow the precursor to transport into a particular liquid phase of the bijel mixture; and
solidifying the precursor-containing liquid phase to form a bijel-templated material (BTM) that is bonded to at least the outer surface of the object.

2. The method of claim 1, wherein the bijel mixture comprises at least a first liquid, a second liquid different than, and partially miscible with, the first liquid, and a plurality of colloidal particles residing at an interface between the two liquids.

3. (canceled)

4. (canceled)

5. The method of claim 1, wherein the object is exposed to the bijel mixture by placing the object in contact with the bijel mixture.

6. The method of claim 1, wherein the object is exposed to the bijel mixture by placing the object in contact with the bijel mixture, the outer surface of the object being chemically activated before being placed into contact with the bijel mixture.

7. The method of claim 1, wherein the solidifying of the precursor-containing liquid phase occurs by polymerization.

8. The method of claim 1 further comprising:
removing unreacted materials by application of a solution that effectuates removal of the unreacted materials.

9. (canceled)

10. The method of claim 1, wherein the BTM comprises a first solid phase and a first non-solid phase, wherein the method further comprises:
filling the first non-solid phase of the BTM with a second precursor;
solidifying the first non-solid phase of the BTM including the second precursor to produce a second solid phase;
removing the first solid phase from the BTM.

11. The method of claim 1, wherein the BTM comprises a first solid phase and a first non-solid phase, wherein the method further comprises:
filling the first non-solid phase of the BTM with a second precursor;
solidifying the first non-solid phase of the BTM including the second precursor to produce a second solid phase.

12.-16. (canceled)

17. A method comprising:
forming a bijel that is exposed to a surface of an object, the bijel including a precursor-containing liquid phase; and
solidifying the precursor-containing liquid phase to form a bijel-templated material (BTM) that is bonded to at least the outer surface of the object.

18. The method of claim 17, wherein the bijel comprises at least a first liquid, a second liquid different than, and partially miscible with, the first liquid, and a plurality of colloidal particles residing at an interface between the two liquids.

19. (canceled)

20. (canceled)

21. The method of claim 17, wherein the solidifying of the precursor-containing liquid phase within the bijel occurs by polymerization.

22. A biomedical device comprising:
an implant for insertion into physiological tissue;
a coating applied to at least a portion of a surface of the implant in contact with the physiological tissue, the coating includes a bijel-templated material (BTM) that is bonded to the surface of the implant.

23. The biomedical device of claim 22, wherein the BTM is formed from at least a bijel mixture including a first liquid, a second liquid different than, and partially miscible with, the first liquid, and a plurality of colloidal particles residing at an interface between the two liquids.

24. (canceled)

25. (canceled)

26. The biomedical device of claim 22, wherein the BTM is formed from at least a bijel mixture including a precursor-containing liquid phase and the precursor-containing phase being solidified.

27. The biomedical device of claim 26, wherein the precursor-containing liquid phase includes a photoinitiator thereby the BTM is formed in response to performing photopolymerization on the bijel mixture including the precursor-containing liquid phase.

28. A method comprising:
forming bijel-templated material (BTM); and
bonding the BTM to an object.

29. The method of claim 28, wherein the BTM is formed from a bijel mixture including at least a first liquid, a second liquid different than, and partially miscible with, the first liquid, and a plurality of colloidal particles residing at an interface between the two liquids.

30. The method of claim 28, wherein the bonding of the BTM to the object comprises (i) adding a precursor-containing liquid phase to the BTM, and (ii) conducting a reaction so that the precursor-containing liquid phase binds the existing solid phase of the BTM directly to at least a surface of the object.

31. The method of claim 28, wherein the BTM comprises a first solid phase and a first non-solid phase, wherein the method further comprises:
filling the first non-solid phase of the BTM with a second precursor;
solidifying the first non-solid phase of the BTM including the second precursor to produce a second solid phase;
removing the first solid phase from the BTM.

32. The method of claim 28, wherein the BTM comprises a first solid phase and a first non-solid phase, wherein the method further comprises:
filling the first non-solid phase of the BTM with a second precursor;
solidifying the first non-solid phase of the BTM including the second precursor to produce a second solid phase.
